(19) United States
(12) Patent Application Publication
(10) Pub. No.: US 2018/0064653 A1
(43) Pub. Date: Mar. 8, 2018

(54) PHARMACEUTICAL COMPOSITIONS OF DIMETHYL FUMARATE

(71) Applicant: HETERO LABS LIMITED, Hyderabad, Telangana (IN)

(72) Inventors: Parthasaradhi Reddy BANDI, Hyderabad, Telangana (IN); Khadgapathi PODILE, Hyderabad, Telangana (IN); Naresh ANAPARTY, Hyderabad, Telangana (IN)

(21) Appl. No.: 15/557,724

(22) PCT Filed: Mar. 14, 2016
(86) PCT No.: PCT/IB2016/051444

§ 371 Date: Sep. 12, 2017

(30) Foreign Application Priority Data
Mar. 17, 2015 (IN) 1310/CHE/2015

(51) Int. Cl. A61K 9/48; A61K 9/28; A61K 9/20; A61K 31/225

(52) U.S. Cl. CPC A61K 9/4891; A61K 9/4808; A61K 31/225; A61K 9/2054; A61K 9/2072; A61K 9/2846

(57) ABSTRACT

The present invention relates to solid oral dosage forms of Dimethyl fumarate. More specifically, the present invention relates to delayed release compositions of Dimethyl fumarate and process for their preparation.

### PRIORITY

This patent application claims priority to Indian patent application number 1310/CHE/2015, filed on Mar. 17, 2015, the contents of which are incorporated by reference herein in their entirety.

### FIELD OF THE INVENTION

The present invention relates to solid oral compositions of dimethyl fumarate. More particularly the present invention relates to delayed release compositions of dimethyl fumarate.

### BACKGROUND OF THE INVENTION

Dimethyl fumarate is an Nrf2 activator described chemically as dimethyl (E) butenedioate with following structural formula:

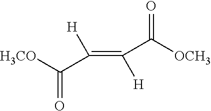

In the United States, dimethyl fumarate is available as 120 mg and 240 mg delayed release capsules under the brand name TECFIDERA® by Biogen Idec Inc.

U.S. Pat. No. 6,509,376 assigned to Biogen Idec disclose composition of dialkyl fumarate in the form of micro-pellets or micro-tablets of size or the mean diameter 5,000μ or less.

WO 2013/076216 A1 assigned to Synthon disclose particle or a plurality of particles of dimethyl fumarate having a D₅₀ particle size distribution between 50 and 1000 μm, wherein each particle is coated by at least one layer comprising a pharmaceutically acceptable pH-dependent entero-resistant polymer.

There remains a need to develop alternative compositions of dimethyl fumarate using simple techniques. Accordingly, inventors of the present invention have developed compositions of dimethyl fumarate that are found to be comparable with that of marketed Tecdifera® capsules.

### SUMMARY OF THE INVENTION

The present invention relates to delayed release solid oral dosage forms comprising Dimethyl fumarate and one or more pharmaceutically acceptable excipients.

One embodiment of the present invention relates to delayed release capsule dosage form comprising a plurality of tablets comprising dimethyl fumarate and one or more pharmaceutically acceptable excipients wherein, the mean diameter of tablet is more than 5 mm.

Another embodiment of the present invention relates to enteric coated tablet composition comprising dimethyl fumarate with a mean diameter of tablet in the range of from 5.2 mm to 6.0 mm.

Yet another embodiment of the present invention relates to composition comprising dimethyl fumarate and one or more pharmaceutically acceptable excipients in a hard gelatin capsule comprising enteric polymers or coated with at least one coating layer wherein, said at least one coating layer delays the release of dimethyl fumarate.

Further embodiment of the present invention relates to method of use of dimethyl fumarate compositions for the treatment of multiple sclerosis in a patient in need thereof.

### DETAILED DESCRIPTION OF THE INVENTION

The present invention relates to delayed release solid oral composition comprising Dimethyl fumarate and one or more pharmaceutically acceptable excipients.

The term “active ingredient” or “active agent” or “drug” used interchangeably, is defined to mean active drug (e.g. dimethyl fumarate), that induce a desired pharmacological or physiological effect.

The term “pharmaceutically acceptable” as used herein means that which is useful in preparing a pharmaceutical composition that is generally safe and non-toxic.

As used in this specification and the appended claims, the singular forms “a”, “an”, and “the” include plural references unless the context clearly dictates otherwise. Thus for example, reference to “a method” includes one or more methods, and/or steps of the type described herein and/or which will become apparent to those persons skilled in the art upon reading this disclosure so forth.

The term “excipient” means a pharmacologically inactive component such as a diluent, a binder, a disintegrant, a glidant, a lubricant, etc of a pharmaceutical product. The excipients that are useful in preparing a pharmaceutical composition are generally safe, non-toxic and are acceptable for human pharmaceutical use. Reference to an excipient includes both one and more than one such excipients.

By the term “solid dosage form” or “dosage form” or “composition” as used herein refers to a solid dosage form suitable for oral administration, such as a tablet, capsule, mini-tablets, spheroids, pellets, granules, pills and the like meant for delayed release.

The term “delayed release” as used herein refers to as that prevents release of the active ingredient in the gastric environment and allows its release in the intestine region.

One embodiment of the present invention relates to delayed release capsule dosage form comprising a plurality of tablets comprising dimethyl fumarate and one or more pharmaceutically acceptable excipients wherein, the mean diameter of tablet is more than 5 mm.

Capsule dosage form according to the present invention is filled with 2 to 4 tablets having mean diameter in the range of 5.2 mm to 6 mm comprising dimethyl fumarate and one or more pharmaceutically acceptable excipients.

The delayed release capsule dosage form according to the present invention comprise a total of 120 mg or 240 mg of dimethyl fumarate.

Pharmaceutical tablet composition according to the present invention comprise excipients selected from diluents, disintegrants, binders, glidants, lubricants, solubilizing agents/surfactants and combinations thereof.

Diluents: Various useful diluents by way of example and without limitation include microcrystalline cellulose, microfine cellulose, powdered cellulose, lactose anhydrous, lactose monohydrate, dibasic calcium phosphate, tribasic calcium phosphate, starch, pregelatinized starch, calcium carbonate, calcium sulfate, magnesium carbonate, magnesium oxide, dextrates, dextrin, dextrose, kaolin, maltodextrin, mannitol, xylitol and sorbitol and the like and combinations thereof.

Binders: Various useful binders by way of example and without limitation include hydroxypropyl cellulose, low-substituted hydroxypropyl cellulose, hydroxypropyl methylcellulose, polyvinyl pyrrolidone, pregelatinized starch, powdered acacia, gelatin, guar gum, carbomers and the like and combinations thereof.

Disintegrants: Various useful disintegrants by way of example and without limitation include sodium starch glycolate, croscarmellose sodium, crospovidone, and the like and combinations thereof.

Glidants: Various useful glidants include but are not limited to colloidal silicon dioxide, other forms of silicon dioxide, such as aggregated silicates and hydrated silica, talc, magnesium silicate, magnesium trisilicate, and the like and combinations thereof.

Lubricants: Various useful lubricants by way of example and without limitation include talc, magnesium stearate, calcium stearate, zinc stearate, stearic acid, palmitic acid, sodium stearyl fumarate, carnauba wax, hydrogenated vegetable oils, mineral oil, polyethylene glycols, and the like and combinations thereof.

Plasticizers: Various useful plasticizers by way of example and without limitation include glyceryl monostearate, triethyl citrate, macrogols, lactic acid, lactic acid acetamide, sorbitol, glycerin, triacetin, acetyl triethyl citrate, acetyl tributyl citrate, dibutyl phthalate, polyvinylpyrrolidone, triethylene glycol, tricresyl phosphate, dibutyl tartrate, ethylene glycol monooleate, palmitic acid, stearic acid, oleic acid, dibutyl sebacate, acetylated monoglycerides, cetyl alcohol and other hydrogenated oils and waxes, as well as polyethylene glycol 300, 400, 600, 1450, 3350 and 8000 and the like or combinations thereof.

Surfactants: Various useful surfactants by way of example and without limitation include sodium lauryl sulfate, docusate sodium, benzekonium chloride, benzethonium chloride and cetrimide, and the like and combinations thereof.

Enteric polymers: Various suitable polymers by way of example and without limitation include methacrylic acid-methyl acrylate copolymer, methacrylic acid-ethyl acrylate copolymer (1:1-2), methacrylic acid-methyl methacrylate copolymer (1:1-2), Poly (methyl acrylate-co-methyl methacrylate-co-methacrylic acid), phthalates, succinates and sodium alginate and the like and combinations thereof.

Another embodiment of the present invention relates to enteric coated tablet composition comprising dimethyl fumarate with a mean diameter of tablet in the range of from 5.2 mm to 6.0 mm.

Enteric coating or the delayed release coating or the coating that delays the release of dimethyl fumarate according to the present invention may be an aqueous or non aqueous coating composition.

Solvents: Various suitable solvents by way of example and without limitation include isopropyl alcohol, dichloromethane, ethanol, methanol, acetaldehyde, acetone, acetonitrile, benzene, N,N-dimethylformamide, ethyl acetate, ethyl ether, ethylene glycol, formaldehyde, isopropanol, methyl n-butyl ketone, methyl ethyl ketone, perchloroethylene, trichloroethane, trichloroethylene; and the like, and combinations thereof; and aqueous solvents such as water.

One another embodiment of the present invention relates to composition comprising dimethyl fumarate and one or more pharmaceutically acceptable excipients in a hard gelatin capsule comprising enteric polymers or coated with at least one coating layer wherein, said at least one coating layer delays the release of dimethyl fumarate.

Another embodiment of the present invention relates to enteric coating comprising polymer selected from one or more of methacrylic acid-methyl acrylate copolymer, methacrylic acid-ethyl acrylate copolymer, methacrylic acid-methylacrylate copolymer, phthalates, succinates and sodium alginate.

The composition being filled into the hard gelatin capsule according to the present invention is in the form of tablets, mini-tablets, spheroids, pellets, granules, pills or plurality of particles, preferably tablets.

Composition of the present invention are prepared by direct compression, dry blending where the composition of the actives and excipients is compacted into a slug or a sheet and then comminuted into compacted granules and then the compacted granules may be subsequently be compressed into a tablet or by wet granulation techniques where active ingredient and some or all of the excipients are blended and then further mixed in the presence of a binder solution, that causes the powders to clump into granules. The granulate is screened and/or milled, dried and then screened and/or milled to the desired particle size. The granulate may then be tableted, or other excipients may be added prior to tableting, such as a glidant and/or a lubricant.

Yet another embodiment of the present invention relates to the process of preparation of tablets by direct compression technique comprising the steps of i) blending dimethyl fumarate, one or more pharmaceutically acceptable excipients, ii) compressing the blend of step (i) to obtain tablets, iii) coating the tablets of step (ii) using enteric coating polymers, iv) filling the tablets of step (iii) in plurality into capsules.

The tablets prepared according to any of the above processes are coated with delayed release coating or enteric coating and filled into capsules.

Tablets of the present invention are optionally coated with a film coating composition.

A film coat on the tablet provides an elegant appearance, protects from moisture and further contributes to the ease with which it can be swallowed.

Further embodiment of the present invention relates to method of use of dimethyl fumarate compositions for the treatment of multiple sclerosis in a patient in need thereof.

Certain specific aspects and embodiments of this invention are described in further detail by the examples below, which are provided only for the purpose of illustration and are not intended to limit the scope of the invention in any manner.

### EXAMPLES

### Example 1

### Delayed Release Capsule Dosage Forms of Dimethyl Fumarate:

| Ingredient | Mg/Capsule |
|---|---|
| Dimethyl fumarate | 240.0 |
| Microcrystalline cellulose | 152.0 |
| Croscarmellose sodium | 36.0 |
| Talc | 4.0 |
| Colloidal silicon dioxide | 4.0 |
| Magnesium stearate | 4.0 |
| Primary Coating | |
| Opadry white enteric coating ^{#} | 4.0 |
| Isopropyl alcohol | q.s |
| Secondary Coating | |
| Acryl-eze white enteric coating ^{$} | 40.0 |
| Purified water | q.s |
| Total | 484.0 |

^{#} Opadry white enteric coating comprises of Methacrylic acid-Methyl Methacrylate Copolymer, Triethyl citrate, Titanium dioxide and Talc.
^{$} Acryl-eze white enteric coating comprises of Methacrylic acid-Ethyl Acrylate Copolymer, Triethyl citrate, Titanium dioxide, Talc, Silica, Sodium bicarbonate and Sodium lauryl sulfate.

### Manufacturing Process:

1. Dimethyl fumarate, microcrystalline cellulose and croscarmellose sodium were sifted through #20,
2. talc, magnesium stearate and colloidal silicon dioxide were sifted through #40 mesh,
3. sifted mixture of step 1 was blended for 10 min,
4. mixture of step 2 was added to step 3 and was mixed for 5 min,
5. blend of step 4 was compressed into tablets of size more than 5 mm,
6. primary coating solution was prepared by dissolving Opadry white enteric coating in isopropyl alcohol,
7. secondary coating solution was prepared by dissolving Acryl-eze white enteric coating in purified water,
8. tablets of step 5 were coated using primary coating solution of step 6,
9. coated tablets of step 8 were coated again using secondary coating solution of step 7,
10. coated tablets were filled into size “0” hard gelatin capsules.

### Example 2

### Delayed Release Capsule Dosage Forms of Dimethyl Fumarate:

| Ingredient | Mg/Capsule |
|---|---|
| Dimethyl fumarate | 120.0 |
| Microcrystalline cellulose | 76.0 |
| Croscarmellose sodium | 18.0 |
| Talc | 2.0 |
| Colloidal silicon dioxide | 2.0 |
| Magnesium stearate | 2.0 |
| Enteric Coating | |
| Opadry white enteric coating ^{#} | 13.0 |
| Triethyl citrate | 2.0 |
| Isopropyl alcohol | q.s |
| Total | 235.0 |

^{#} Opadry white enteric coating comprises of Methacrylic acid-Methyl Methacrylate Copolymer, Triethyl citrate, Titanium dioxide and Talc.

### Manufacturing Process:

1. Dimethyl fumarate, microcrystalline cellulose were sifted through #20 mesh and blended,
2. croscarmellose sodium was sifted through #20 mesh and added to blend of step 1 and blended,
3. talc, colloidal silicon dioxide were sifted through #40 mesh, added to blend of step 2 and blended for 10 minutes,
4. magnesium stearate was sifted through #60, added to material of step 3 and lubricated for 5 minutes,
5. blend of step 4 was compressed into tablets of size more than 5 mm,
6. coating solution was prepared by dissolving Opadry white enteric coating and triethyl citrate in isopropyl alcohol,
7. tablets of step 5 were coated using enteric coating solution of step 6,
8. coated tablets of step 7 were filled into size “0” hard gelatin capsules.

### Example 3

### Delayed Release Capsule Dosage Forms of Dimethyl Fumarate:

| Ingredient | (% w/w) |
|---|---|
| Dimethyl fumarate | 60.67 |
| Microcrystalline cellulose | 34.04 |
| Croscarmellose sodium | 4.25 |
| Talc | 0.52 |
| Colloidal silicon dioxide | 0.52 |
| Total | 100 |

### Manufacturing Process:

1. Dimethyl fumarate, microcrystalline cellulose and croscarmellose sodium were sifted through #20,
2. talc and colloidal silicon dioxide were sifted through #40 mesh,
3. sifted mixture of step 1 was blended for 10 min,
4. mixture of step 2 was added to step 3 and was mixed for 5 min,
5. blend of step 4 was filled into enteric coated size “0” hard gelatin capsules.

1-10. (canceled)

11. A delayed release capsule dosage form comprising:
a plurality of tablets having a mean diameter of more than 5 mm,
wherein the tablets comprise dimethyl fumarate and one or more pharmaceutically acceptable excipients.

12. The dosage form according to claim 11 comprising 2 tablets, wherein the mean diameter is 5.2 mm to 6.0 mm.

13. The dosage form according to claim 11 comprising 3 tablets, wherein the mean diameter is 5.2 mm to 6.0 mm.

14. The dosage form according to claim 11 comprising 4 tablets, wherein the mean diameter is 5.2 mm to 6.0 mm.

15. The dosage form according to claim 11, wherein the amount of dimethyl fumarate is 120 mg.

16. The dosage form according to claim 11, wherein the amount of dimethyl fumarate is 240 mg.

17. The dosage form according to claim 11, wherein the tablets are coated with enteric coating polymers.

18. The dosage form according to claim 11, wherein the tablets comprise one or more excipients selected from the group consisting of microcrystalline cellulose, croscarmellose sodium, colloidal silicon dioxide, talc and a combination thereof.

19. A composition comprising enteric coated tablets having a mean diameter of 5.2 mm to 6.0 mm, wherein the tablets comprise dimethyl fumarate and one or more pharmaceutically acceptable excipients.

20. The composition according to claim 19, comprising a polymer selected from the group consisting of methacrylic acid-methyl acrylate copolymer, methacrylic acid-ethyl acrylate copolymer, methacrylic acid-methylacrylate copolymer, phthalates, succinates and sodium alginate.

21. The composition according to claim 19, wherein the tablets are filled into a capsule.

22. A method of obtaining the composition according to claim 21, comprising:
(a) preparing the tablet by direct compression technique comprising the steps of:
(i) blending dimethyl fumarate and one or more pharmaceutically acceptable excipients,
(ii) compressing the blend of step (i) to obtain tablets, and
(iii) coating the tablets of step (ii) with enteric coating polymers, and
(b) filling a capsule with a plurality of tablets obtained in step (iii).

23. A composition comprising dimethyl fumarate and one or more pharmaceutically acceptable excipients in a hard gelatin capsule, wherein the capsule comprises enteric polymers or coated with at least one enteric coating layer which delays the release of dimethyl fumarate.

24. A method of treating patients with relapsing forms of multiple sclerosis, comprising administering to a patient in need thereof the capsule dosage form according to claim 11.
